(19) United States
(12) Patent Application Publication
(10) Pub. No.: US 2016/0270725 A1
(43) Pub. Date: Sep. 22, 2016

(54) WEARABLE LACTATE THRESHOLD MONITOR

(71) Applicants: Jeffrey Allen Gray, Sudbury, MA (US); Nathan Zamarripa, Kittery Point, ME (US)

(72) Inventors: Jeffrey Allen Gray, Sudbury, MA (US); Nathan Zamarripa, Kittery Point, ME (US)

(21) Appl. No.: 15/070,412

(22) Filed: Mar. 15, 2016

Related U.S. Application Data
(60) Provisional application No. 62/171,069, filed on Jun. 4, 2015.
(60) Provisional application No. 62/133,730, filed on Mar. 16, 2015.

(51) Int. Cl. A61B 5/00; A61B 5/024; A63B 24/00; A61B 5/055

(52) U.S. Cl. CPC A61B 5/4884; A61B 5/055; A61B 5/0022; A61B 5/681; A61B 5/024; A63B 24/0062

(57) ABSTRACT

A wearable device for monitoring a lactate threshold can include at least one magnet configured to provide a static magnetic field to an anatomical region, a power supply, and a radio frequency (RF) module connected to the power supply. The RF module can provide pulsating RF signals across the static magnetic field and emit energy into, and receive response signals from, the anatomical region over a period of time. The response signals can enable detection of a change in H⁺ concentration in the anatomical region, the change enabling detection of the lactate threshold. The wearable device can be used during a variety of exercises at selectable anatomical locations.

### RELATED APPLICATION

This application claims the benefit of U.S. Provisional Application No. 62/171,069, filed on Jun. 4, 2015 and U.S. Provisional Application No. 62/133,730, filed on Mar. 16, 2015. The entire teachings of the above applications are incorporated herein by reference.

### BACKGROUND OF THE INVENTION

Modern athletic training programs are based upon exertion expressed as a percentage of an individual's lactate threshold. Due to the variation in efficiency of muscle groups that may be engaged during various activities, the lactate threshold of an individual also varies depending upon the activity in which the individual is engaging. For example, an individual's lactate threshold may be higher when that individual runs, as opposed to when he or she swims or bikes.

Currently available wearable training devices for detecting an individual's lactate threshold typically emit light of specific wavelengths, which are shone through the skin and into muscle tissue to continually measure concentrations of CO₂ and/or O₂ in the individual's muscle. This data can then be used to infer the individual's lactate threshold. These devices have several shortcomings. Light-based devices require that they be utilized with a larger muscle mass, such as the calf muscle, in order to obtain measurements of CO₂ and/or O₂. Additionally, light-based devices require direct contact with the skin of the individual at the location of the muscle mass. These limitations can make the device bulky, uncomfortable, or impractical for some sports and for some athletes.

The measurements of CO₂ and/or O₂ obtained by light-based devices represent local changes in the muscle being measured, from which a lactate threshold may be inferred. As such, the device must be positioned at a muscle that is being engaged during the activity. Use of these devices is typically limited to sports such as running and cycling, in which an individual is heavily engaging his or her calf muscles and which permit contact of the device with the area being measured.

As such, there is a need for an improved training device that can be used during a variety of exercises and provide flexibility with respect to an anatomical location at which the device may be worn.

### SUMMARY OF THE INVENTION

A wearable device for monitoring a lactate threshold can include at least one magnet configured to provide a static magnetic field to an anatomical region, a power supply, and a radio frequency (RF) module connected to the power supply. An RF module may contain a signal transmitting coil, a signal receiving coil, or an integrated transceiver (which can perform both transmit and receive functions), to send and receive radio wave signals as well as radio wave responses from the anatomical region over a period of time. The RF module can be configured to provide pulsating RF signals across the static magnetic field and emit energy into the anatomical region over the period of time, and further configured to receive response signals from the anatomical region in response to the energy emitted into the anatomical region over the period of time. The response signals can enable detection of a quantity of H⁺ protons in the anatomical region. Continuous monitoring of the quantity of H⁺ protons in the anatomical region allows a change in this value to be detected. A change in concentration of H⁺ can enable detection of a lactate threshold of a wearer of the device. For example, a sharp increase in a detected concentration of H⁺ can indicate that the wearer's lactate threshold has been reached.

A method of measuring lactate threshold can include exposing an anatomical region to a magnetic field, the magnetic field provided by at least one magnet within a wearable device. The magnet may be either “open,” which may not fully encompass the anatomical region, or “closed,” where the magnet may fully encircle the anatomical region. The method can further include emitting a pulsating RF signal into the anatomical region over a period of time, the RF signal provided by an RF signal generator and an RF transmit coil, and receiving a response signal from the anatomical region by at least one RF receive coil or integrated transceiver. Based upon the response signal, concentrations of H⁺ within the anatomical region over the period of time can be measured, and a lactate threshold based upon a detected change in the concentration of H⁺ within the anatomical region can be determined. A change in H⁺ concentration within an anatomical region can indicate a systemic response in the individual.

The period of measurement time can be any period of time from about one minute to about two hours. For example, the period of measurement time can be at least one minute, at least five minutes, at least thirty minutes, or at least one hour. The wearable device can further include a processing component and software for calculating a moving average of H⁺ concentration within the anatomical region. A processing component can also adjust the energy that is emitted into the tissue based on a detected response signal. The components of the device, including the magnet, RF signal generator, and RF coil(s), can be contained in a housing that may have an ergonomic design for comfort of the wearer. A Faraday cage surrounding the componentry can be included, which can thereby minimize interference from externally produced radio frequency waves. The magnet can have a magnetic flux density of 0.1 T to 2.0 T, or 0.25 T to 0.5 T. The wearable device can be wristwatch, anklet, or armband. The power supply can be a rechargeable or replaceable battery. The wearable device can further include a wireless transmission component for transmitting H⁺ concentration data to a server in a network, and a display for displaying a detected change in H⁺ concentration. The wearable device can also include components for other functions, such as a heart rate monitor, pedometer, altimeter, thermometer, and clock.

A system of measuring lactate threshold can include a wearable device comprising a magnet, power supply, RF signal generator, RF transmit coil, and RF receive coil or integrated transceiver. The system can further include a wireless transmission component for transmitting H⁺ concentration data over a network and a server in the network having a software routine, which, when executed, causes the server to receive H⁺ concentration data. The server can further calculate athletic training zones based on percentages of H⁺ concentration data and transmit athletic training zone information to a device. The device to which the athletic training zone information is transmitted can be a personal computer, tablet, smartphone, or the wearable device. The wearable device of the system can include a digital display configured to render a display of the athletic training zone information transmitted to the wearable device from the server. The wearable device can include a data storage device configured to store H⁺ concentration data and further configured to connect with a personal computer, tablet, or smartphone. The wearable device can also include a display including colored lights, with each color respectively indicating below, at, or above threshold conditions, and thereby acting as signals to the user of the current degree of physical activity.

A wearable device for monitoring a lactate threshold can include at least one magnet configured to provide a static magnetic field to an anatomical region, a power supply, and a radio frequency (RF) module connected to the power supply and configured to provide pulsating RF signals across the static magnetic field. The RF module can emit energy into, and receive response signals from, the anatomical region over a period of time. The response signals can enable detection of a change in H⁺ concentration in the anatomical region, and the change in H⁺ concentration can enable detection of the lactate threshold.

The period of time can be between thirty seconds and one hour, or between an hour and twenty-four hours. A processing component included in the device can be coupled to the RF module and configured to calculate a moving average of H⁺ concentration within the anatomical region and/or configured to adjust the energy emitted into the anatomical region based on a detected response signal. A processor coupled to the RF module can also be configured to generate a representation of a change in H⁺ concentration, and the device can include a display configured to display a detected change in H⁺ concentration. The display can be further configured to display athletic training zone information transmitted to the wearable device from a server.

The RF module can include an RF signal generator, RF transmit coil, and RF receive coil, which can be included with the power supply within a housing. The power supply can be a rechargeable battery. The device can further include a Faraday cage encapsulating the magnet and the RF module. The device can also include a wireless transmission component communicatively coupled to the RF module and configured to for transmit H⁺ concentration data to a server via a network

The magnet can be an open or closed magnet and can have a magnetic flux density of 0.1 T to 2.0 T, or of 0.25 T to 0.5 T. The device can be in the form a wristwatch, anklet, or armband.

A data storage device can be included in the device and configured to store H⁺ concentration data. A data transmitter module can also be included and configured to connect with a personal computer to download the stored H⁺ concentration data. The wearable device can also include features of a heart rate monitor, pedometer, altimeter, thermometer, and/or clock.

A method of determining a lactate threshold can include applying a magnetic field to an anatomical region, the magnetic field provided by at least one magnet within a wearable device, and emitting pulsating RF signals into the anatomical region over a period of time, the RF signals provided by an RF module. The method can further include receiving response signals from the anatomical region by the RF module, measuring concentrations of H⁺ within the anatomical region over the period of time based upon the response signals, and determining a lactate threshold based upon a detected change in the concentration of H⁺ within the anatomical region.

The method can further include operating the RF module within a housing and maintaining a position of the housing within proximity of a limb. Transmitting measurements of the concentration of H⁺ within the anatomical region to a server via a network and displaying a detected change in the concentration of H⁺ can also be included. The period of time can be between thirty seconds and one hour, or between an hour and twenty-four hours.

A system includes a server in a network, the server having a software routine operable thereon, the software routine, when executed, causes the server to receive H⁺ concentration data from a wearable device, calculate athletic training zones based on percentages of the H⁺ concentration data, and transmit athletic training zone information to the wearable device or a different device. The software routine can be further configured to cause the server to transmit the athletic training zone information to a personal computer, tablet, smartphone, or personal digital assistant.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a graph illustrating an example of blood lactate data as a function of running speed.

FIG. 2 is a cross-sectional view of an example of a wearable lactate threshold monitor.

FIG. 3 is schematic of magnetic resonance components and circuitry contained within an example of a wearable lactate threshold monitor.

FIG. 4A is an illustration of a wearable lactate threshold monitor applied to a wrist and illustrating an interaction between a static magnetic field applied by the device and hydrogen protons of affected tissue/blood.

FIG. 4B is an illustration of a wearable lactate threshold monitor of FIG. 4A with a radio frequency pulse being emitted by the device and further illustrating hydrogen protons affected by the pulse spinning to a high energy state.

FIG. 4C is an illustration of the wearable lactate threshold monitor of FIGS. 4A and 4B, following the application of the radio frequency pulse, with affected hydrogen protons relaxing, spinning back to a low energy state.

FIG. 5 is a schematic diagram illustrating use of a wearable lactate threshold monitor in conjunction with other networked devices.

FIG. 6 is chart illustrating a series of training zones.

FIG. 7 is a block diagram illustrating a data processing sequence of a system including a wearable lactate threshold monitor.

FIG. 8 is a schematic view of a computer network environment in which embodiments of the present invention may be deployed.

FIG. 9 is a block diagram of computer nodes or devices in the computer network of FIG. 8.

### DETAILED DESCRIPTION OF THE INVENTION

A description of example embodiments of the invention follows.

A wearable, magnetic resonance (MR) device for monitoring a lactate threshold is provided. MR-based wearable devices can directly detect a concentration, or an increase in concentration, of hydrogen protons in a subject's blood, and indicate to the subject when the lactate threshold has been reached. Additionally, an MR-based wearable device provides added flexibility and convenience for use of the device with sports other than running or cycling.

### Lactate Threshold

The process of cellular respiration occurs in all organs and all cells throughout the body. In particular, blood cells perform the metabolic reactions of cellular respiration and deliver resulting fuel to muscle tissue. The process of cellular respiration converts glucose, which is present in the extracellular fluid surrounding blood cells, into Adenosine Triphosphate (ATP) via glycolysis. This reaction requires the availability of oxygen (O₂) and produces waste products, including carbon dioxide (CO₂) and water (H₂O). Waste products are expelled through cell membranes and back into the extracellular fluid.

As blood travels through the body, it passes through muscle tissue, where ATP is extracted and consumed by the muscle tissue during muscle contractions, which stimulate motion. The more intense a muscle contraction, the more ATP is consumed. In a normal state (e.g., at rest, or at lower or moderate levels of physical exertion), the rate of ATP production by the primary metabolic pathway (e.g., aerobic respiration in the presence of oxygen) meets or exceeds the demands made by muscle tissue. As activity level increases, the demand for ATP also increases. At some point, the rate of glycolysis exceeds the availability of oxygen in the system and aerobic respiration is no longer sufficient to adequately supply ATP to the muscles. At this point, a secondary system kicks in, which allows the cells to continue to produce ATP in the absence of oxygen.

The secondary system or pathway (e.g., anaerobic respiration), also referred to as fermentation, allows the production of ATP without oxygen. Fermentation also causes the production of byproducts, one of which is lactate. Lactate serves the body by acting as a buffer to acidic hydrogen protons (H⁺), which build up in the body as a result of intense exercise. In a normal state, the CO₂ and H₂O that are produced during glycolysis are removed via a bicarbonate buffering reaction. More specifically, CO₂ and H₂O combine to become carbonic acid (H₂CO₃). Carbonic acid is a weak acid and quickly dissociates to become bicarbonate (HCO₃⁻) and hydrogen (H⁺). The bicarbonate buffering reaction is regulated by the lungs, which expel excess CO₂, the kidneys, which expel excess H₂O and HCO₃⁻, and the pancreas, which produces HCO₃⁻. When these systems (e.g., the lungs, kidneys, and pancreas) cannot keep up with an increased production of H⁺, the pH of the blood drops, becoming acidic.

Upon an increase in blood acidity, lactate concentration spikes to buffer the increased concentration of H⁺. A spike in lactate concentration indicates that the body cannot generate sufficient ATP via the primary metabolic pathway to support the intensity and rate of muscular contraction demanded by the nervous system and that the body is utilizing the secondary metabolic pathway for a significant part of ATP production. The point at which the system can no longer clear excess waste products (e.g., CO₂ and H₂O) from the body at the same rate of production of the waste products is the point at which lactate begins to accumulate in the blood stream and is referred to as the “lactate threshold.”

The lactate threshold can also be referred to as the lactate turnpoint and the anaerobic threshold. Lactate thresholds vary amongst individuals and, within a given individual, from one sport to another. As the anaerobic metabolism of glucose is not sustainable for long periods of time, athletes can benefit from understanding their lactate threshold to understand their sustainable level(s) of performance.

Lactate thresholds are typically measured through use of a blood test, with blood samples drawn from a subject at consistent time intervals, for example, about every four minutes. Each sample is analyzed to determine the concentration of lactate contained in the blood at a given point in time. By plotting lactate concentration versus time, the point in time at which the lactate concentration increases can be quantitatively determined. This information can then be correlated to a performance parameter, which the athlete can then utilize to understand the level of effort at which he or she reached the lactate threshold. Typically, time points are correlated to an amount of power (e.g., in watts) produced by the athlete. Alternatively, the time points can be correlated to the athlete's heart rate or pace/speed.

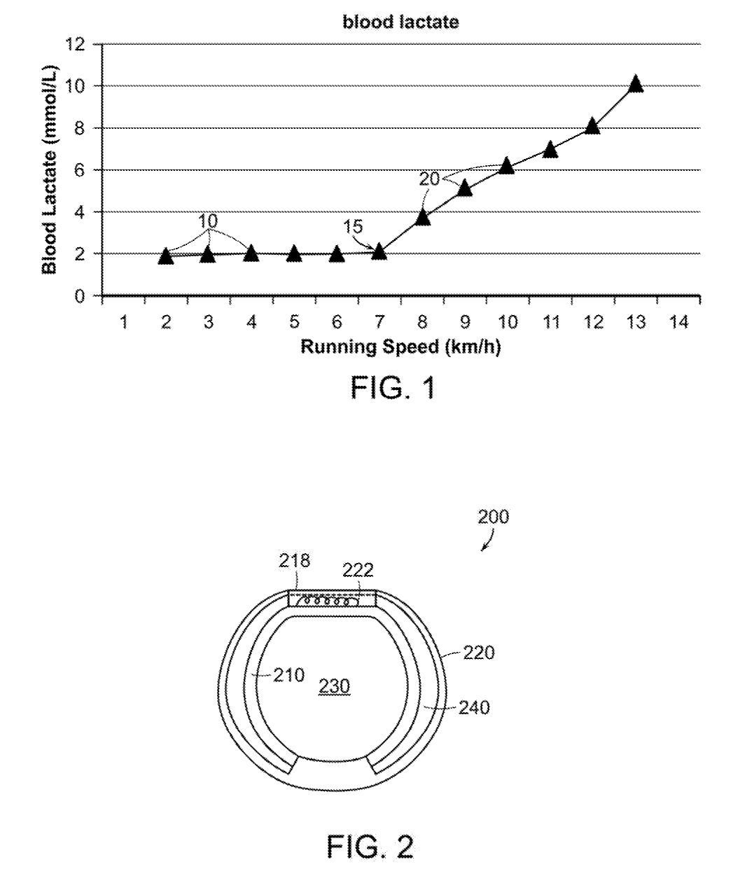

For example, a graph including a series of data points representing lactate levels of an example subject is shown in FIG. 1. The lactate level data is plotted against the subject's running speed. The lactate data represents lactate concentrations as measured from blood samples taken at multiple time points, over which the subject ran at an ever increasing pace. As shown in FIG. 1, lactate levels 10 remain generally constant until the subject's running pace reaches approximately 7 km/h. As the subject's pace increases beyond this level of performance, the lactate levels 20 in the subject's blood also increase. The inflection point 15 represents the speed at which, or, alternatively, the point in time that, the subject reached his lactate threshold. By understanding the stress level in terms of, for example, wattage, heart rate, or speed at which the lactate threshold is reached, the athlete can then understand the level of performance that he or she can maintain for significant periods of time (e.g., minutes or hours, depending upon the activity). In the example of FIG. 1, the athlete can recognize that running speeds greater than 7 km/h are not sustainable, as the athlete's body begins to rely more heavily on the secondary metabolic pathway for ATP production.

### MR-Based Wearable Lactate Threshold Monitors

MR technology, or Nuclear Magnetic Resonance (NMR) technology, can provide a direct measurement of a concentration of H⁺ in an individual's blood, and thereby provide an improved method of detection of the individual's lactate threshold over light-based devices, which indirectly calculate lactate levels based on measured concentrations of CO₂ and/or O₂. Additionally, the effect of lactate concentration spiking as a result of strenuous exercise is systemic. Accordingly, a measurement of H⁺ concentration can be obtained from any anatomical region of the individual by an MR-based wearable device. The wearable device need not be worn at, for example, a calf muscle, as required by light-based devices, which measure local changes in waste product concentrations. An MR-based wearable device can provide added flexibility because it may be worn at other, more comfortable or more convenient locations on the body and it may be worn for activities other than running and cycling.

A cross-sectional view of a wearable device for monitoring a lactate threshold is illustrated in FIG. 2. The wearable device 200 includes at least one magnet 210 located in a housing 220 and configured to provide a static magnetic field to an anatomical region. The device also includes a coil 222, which can function as an RF antenna (e.g., a transmit/receive coil). The magnetic field can be applied across an anatomical region, for example, a limb. Alternatively, the magnetic field can be applied into an anatomical region, for example into a region just below the skin or epidermis and into to the capillaries. As shown in FIG. 2, the wearable device 200 includes a space 230 for the anatomic region that the device 200 may act upon. The device 200 can be threaded over a wrist (not shown) and worn as a cuff or bracelet, with the magnet 210 partially encircling the wrist and applying a static field magnetic into or across the wrist. Wearable devices can include open magnets, such as magnet 210 illustrated in FIG. 2, or closed magnets, such as magnets configured to fully encircle an anatomical region. Alternatively, a wearable device can include several magnets located in various positions within, or attached to, the wearable device.

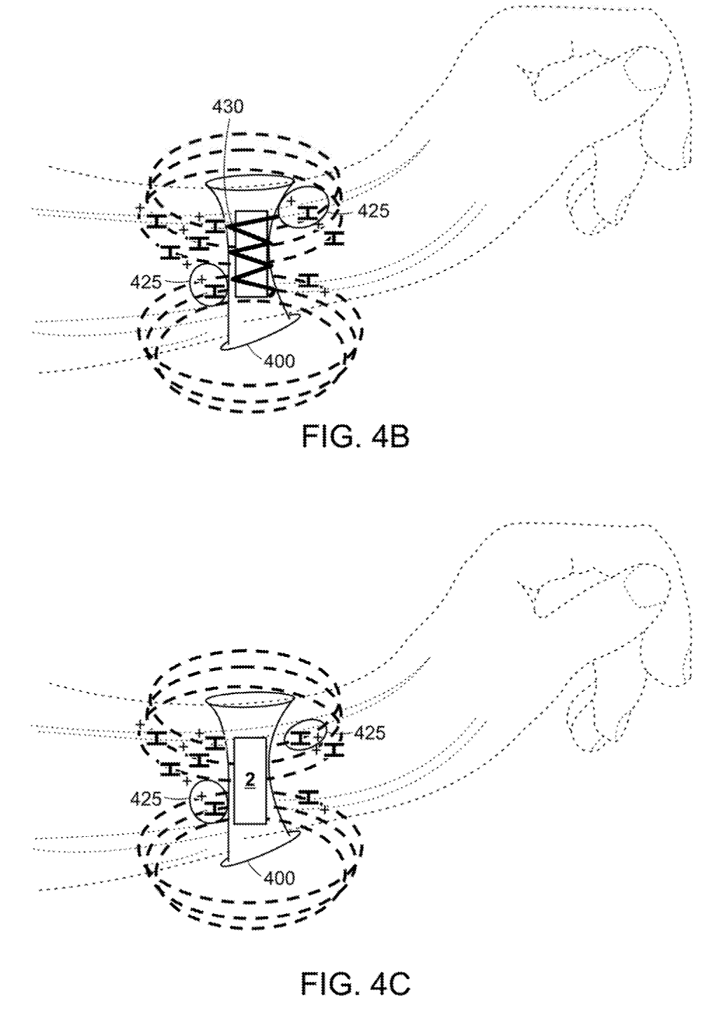

The magnetic field strength, or magnetic flux density, of a magnet included in a wearable device can be of about 0.1 Tesla (T) to about 2.0 T, or of about 0.25 T to about 0.5 T. In a particular embodiment, a magnet has a magnetic flux density of about 0.25 T. The static magnet provides a magnetic field that passes through or into tissue/bone in the vicinity of the magnet, as further described with respect to FIGS. 4A-4C. Hydrogen protons (H) present in the tissue align in the direction of the magnetic field.

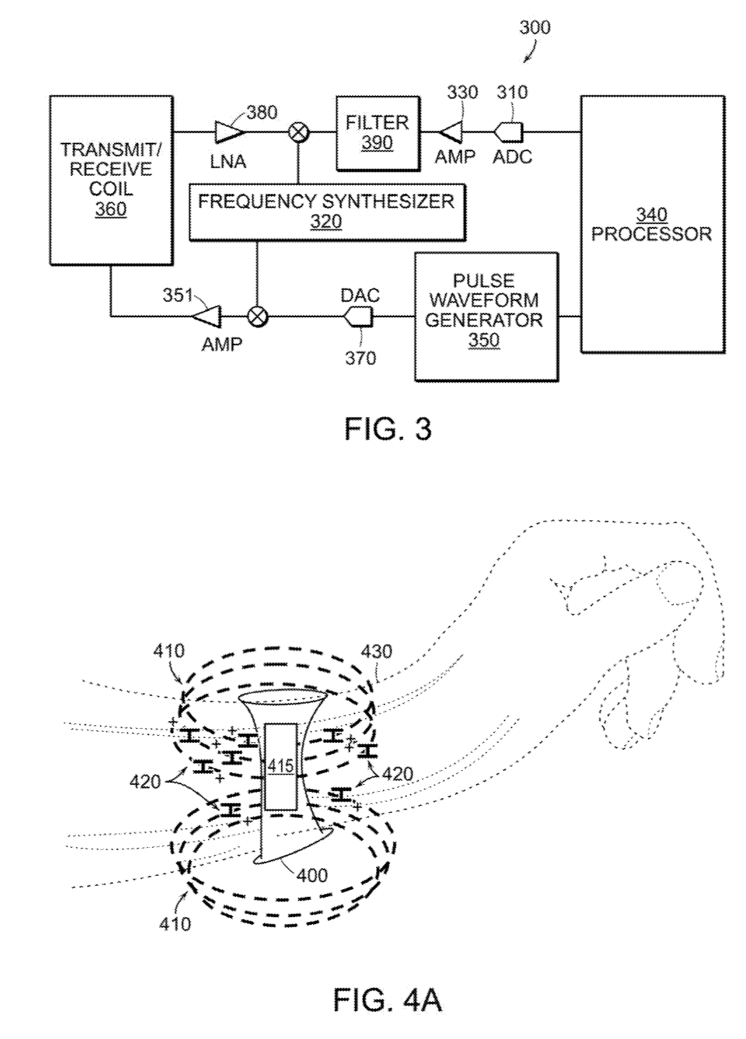

The wearable device 200 can include components (e.g., magnet 210, radiofrequency (RF) transmit/receive coil 360 (FIG. 3), and other circuitry) within a housing 220. For example, the components can be encapsulated in a soft plastic material, such as urethane, silicon, or neoprene. Alternatively, or in addition, the housing can include an internal or external rigid or metal compartment to provide protection to some or all of the components. In a particular embodiment, a Faraday cage 240, which is optional, can be included and can house the at least one magnet and related componentry (FIG. 2) to block external electrical fields, radio frequency waves, and other interferences. The housing 220 can be configured to be worn circumferentially around a limb, such as a bracelet, wristwatch, anklet or armband. The device 200 may be shaped similar to a wristwatch or bracelet, which may be wrapped against or slipped over a hand. The device 200 can have the ability to be closely secured against the anatomical region of interest.

To provide a radio frequency (RF) pulse into the anatomic area, wearable devices can include a radio frequency (RF) signal generator connected to a power supply. The wearable device can further include at least one RF transmit coil connected to the RF signal generator. The RF signal generator, combined with any coils, may collectively be referred to as an “RF module.” An RF module can contain a signal transmitting coil and a signal receiving coil, or an integrated transceiver (which can perform both transmit and receive functions), to send and receive radio wave signals as well as radio wave responses from an anatomical region over a period of time.

The RF module can be configured to provide pulsating RF signals across the static magnetic field and emit energy into the anatomical region over the period of time, and further configured to receive response signals from the anatomical region in response to the energy emitted into the anatomical region over the period of time. The response signal can enable detection of a quantity of H⁺ protons in the anatomical region. Continuous monitoring of the quantity of H⁺ protons in the anatomical region allows a change in this value to be detected. A change in the concentration of H⁺ can enable detection of a lactate threshold of a wearer of the device. For example, a sharp increase in a detected concentration can indicate that the wearer's lactate threshold has been reached. The device thus enables detection of the wearer's lactate threshold.

FIG. 3 is a schematic illustrating components of an example of an RF module 300 of an MR-based wearable lactate threshold monitor. A processor 340 manages data receipt and is also connected to a pulse/waveform generator 350, alternatively referred to as an RF signal generator, which can provide a frequency signal to the transmit/receive coil 360, alternatively referred to as an integrated transceiver. A digital-to-analog converter (DAC) 370 can be wired in between the pulse/waveform generator 350 and the transmit/receive coil 360. The DAC converts the digital signal from the pulse/waveform generator 350 into an analog signal, which can be read by the transmit/receive coil 360 and turned into a radio frequency (RF) wave emitted by the transmit/receive coil 360. The analog signal from the DAC to the transmit/receive coil (e.g., a “send signal”) may be increased in magnitude by an amplifier 351.

The RF wave emitted from the transmit/receive coil 360 can travel through the atmosphere surrounding the device. The frequency of the RF wave can be of about 2 MHz to about 60 MHz to detect hydrogen content. In a particular embodiment, the frequency of the RF wave is of about 40 MHz to about 54 MHZ, or of about 42.58 MHz, which is s typical resonant frequency of H⁺ protons in blood. The energy from an emitted RF wave can be absorbed by tissue or bone in the general vicinity of the device.

In particular, H⁺ protons present in the tissue absorb the RF energy of the emitted wave, causing them to align directionally with the RF wave. The components that trigger the transmit signal (processor 340, pulse/waveform generator 350, transmit/receive coil 360, DAC 370) can be activated for a short period of time (e.g., 10 ms-2000 ms), at the end of which, the energy absorbed by the H⁺ protons is released and returned to the device in the form of another RF wave. The transmit/receive coil 360 can receive the energy released from the H⁺ proton and a signal (e.g., a “receive signal”) can be returned to the processor 340 via circuitry which can including a low noise amplifier (LNA) 380, other filters 390 (e.g., noise reduction filters), and other amplifier(s) 330, and an analog-to-digital converter (ADC) 310. If the send and receive signals utilize independent circuits, then they can be connected with a frequency synthesizer 320 to minimize overlap between the two signals.

FIGS. 4A-4C illustrate an example use of a wearable lactate threshold monitor. As shown in FIG. 4A, a wearable device 400 is worn on a wrist 430. The wearable device 400 includes a static field magnet 415. For illustration, the static field magnet 415 is shown exposed in FIG. 4A; however, the static field magnet 415 can be contained within the device 400, for example, within straps of a wristwatch or within an open or closed band of elastic, silicone, or other material of the wearable device. Magnetic flux, represented by dashed lines 410, is emitted from the static field magnet 415 and affects the tissue and blood that is in the vicinity of the device. In particular, hydrogen protons 420 in the tissue and blood can align with the magnetic flux from the static magnet.

FIG. 4B illustrates the wearable device 400 during emission of a radiofrequency signal, represented by wave 430, from an RF coil embedded in the device 400 (e.g., transmit/receive coil 360 of FIG. 3). Some hydrogen protons 425 in the vicinity of the device 400 are shown to be affected by the RF wave energy, absorbing it and spinning to a high energy state. As shown in FIG. 4B, hydrogen protons 425 align in the opposite direction of their low energy state.

FIG. 4C illustrates the wearable device 400 following emission of the radiofrequency pulse. As illustrated, the RF signal 430 is no longer generated and hydrogen protons 425, which had absorbed the RF energy (FIG. 4B), subsequently release the energy and relax to their low-energy positions, which are still aligned with the magnetic flux of the static field magnet. As the hydrogen protons 425 release their energy, an RF coil embedded in the device 400 (e.g., transmit/receive coil 360 of FIG. 3) receives the energy and a signal processor embedded in the device (e.g., processor 340 of FIG. 3) counts the signals received from the protons 425 returning to their low-energy states. As illustrated in FIG. 4C and for example, a count of two results. Affected hydrogen protons 425 are shown in FIGS. 4A-4C for illustration purposes. In use, an actual count of hydrogen protons that are detected returning to their low-energy may be much greater and may vary depending on the area of the anatomical region being measured.

The wearable device can transmit and receive RF energy signals from the anatomical region over a long period of time. For example, a continuous cycling of transmitting and receiving energy signals can occur over the period of time that a wearer of the device exercises. The period of time can be at least one minute or at least one hour. The period of time can be up to several hours, such as 10, 12, 14, or 20 hours. The power supply of the wearable device can be a rechargeable or replaceable battery. The battery can provide adequate power for long-term use of the device, for example, at least up to 10, 12, 14, or 20 hours, without requiring recharging or replacement. Other power sources, such as hydrogen fuel power supplies, may alternatively be used.

The wearable device can further include a processing component (e.g., processor 340, or a secondary processor) for calculating a moving average of H⁺ concentration during wear. The use of moving average can ensure accuracy of the H⁺ concentration measurement. The moving average H⁺ concentrations are continuously captured. When a significant increase in average H⁺ concentration occurs as compared to a preceding average, there is a direct indication that a lactate threshold has been reached. It should be understood that H⁺ concentration measurements (data points) may be gathered over more or less time. Likewise, more or fewer data points over the given amount of time may be used in the calculation of the moving average. The processing component can further calculate additional information, including training zone information.

The wearable device can further include a processing component for adjusting the energy emitted into the tissue of the anatomical region based on a detected response signal. As such the RF input signal can be increased, decreased, or modulated as necessary during use of the device in order to obtain appropriate response signals.

The device can include a storage medium for storing H⁺ concentration data over the duration of a workout. The device can include a port for connecting the internal storage medium to a personal computing device. The H⁺ concentration data can be downloaded to the personal computing device. The H⁺ concentration data can be further manipulated by an application running locally on the personal computing device.

The device can further include a wireless transmitter for transmitting H+ concentration data to a server in a network, such as a cloud network. The device can also include a wireless receiver for receiving information, such as training zone information, back from the server. The server can host an application that calculates training zones for the individual based upon the H⁺ concentration measurements.

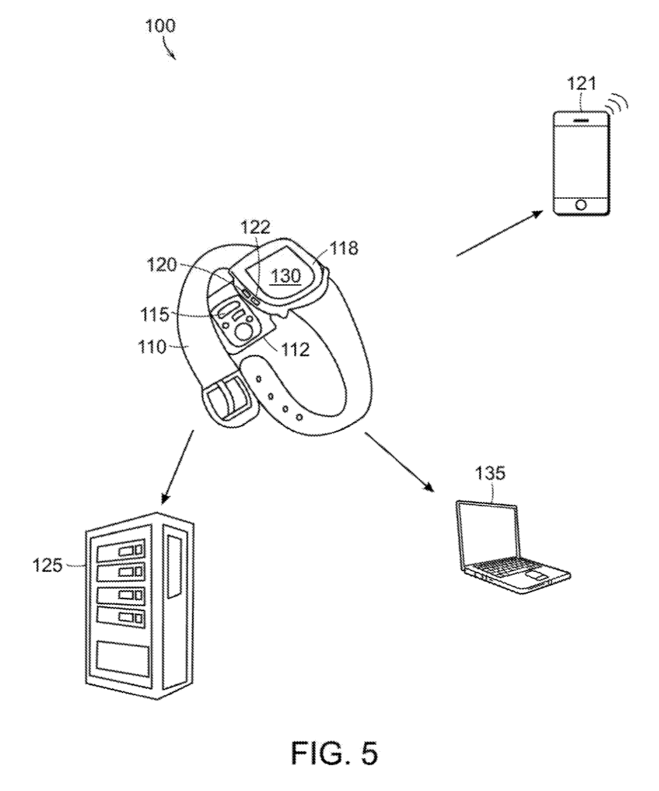

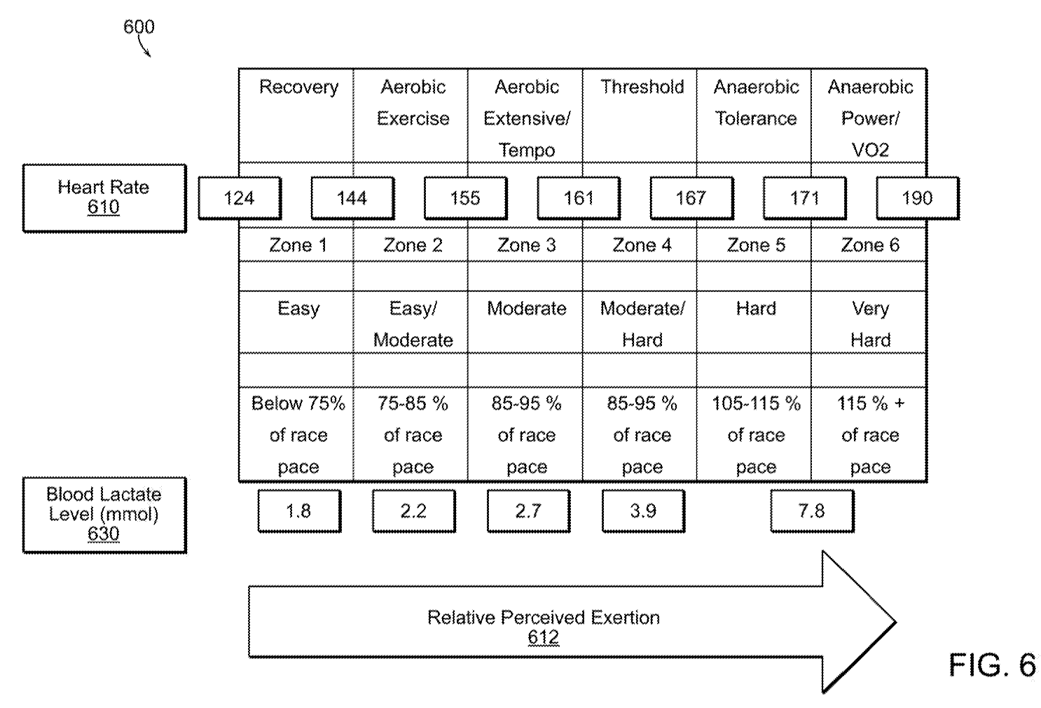

FIG. 5 is illustrates use of a system 100 with a wearable lactate threshold monitor 110. A wearable lactate threshold monitor 110 is shown as a wristwatch with a back panel 112 of the device shown in an exploded view. The wearable device 110 includes a display 130, wireless transmitter 115, and ports 120, 122. Components and other circuitry (e.g., components of FIG. 3, power source, storage medium, heart rate monitor, etc.) can be contained within the device, for example, as part of the back panel 112 and/or within a housing 118. The display 130 can be a simple, or analog, display, such as one or two lights (e.g., a light emitting diode (LED)), for indicating if a lactate threshold of the wearer is met. For example, a red light may indicate that the wearer is functioning beneath his lactate threshold, and a green light may indicate that the wearer is functioning above his lactate threshold, or vice versa. Alternatively, the wearable device can include a digital display. The digital display can present additional information to the user, such as training zone information (e.g., training zones 600 of FIG. 6). The device 110 can be a rechargeable device. The port 122 can be, for example, a battery recharging port.

A wireless transmitter 115 can transmit H⁺ concentration data to a mobile device 121, such as a smartphone (e.g., an iPhone® or Android™), or to a remote server 125. For example, wireless transmitter can transmit data wirelessly via Bluetooth®, ANT+, or other wireless protocols. Additional computations can be performed by the mobile device 121 and/or the remote server 125. Information can be wirelessly transmitted back to the wearable device 110 and displayed on the display 130. Alternatively, or in addition to wireless transmission, wearable device 110 can be connected to a personal computer 135, such as a desktop or laptop, by a wired connection through the port 120. The port 120 can be, for example, a standard mini-USB data port or other port enabling a wired connection to a personal computer 135 or to a mobile device 121. Additional computations can be performed by the personal computer 135. Additional computations that can be performed by the mobile device 121, remote server 125, and/or personal computer 135 include analysis of H⁺ concentration data and calculation of athletic training zones (e.g., training zones 600 of FIG. 6). Alternatively, or in addition, training zones can be calculated by a local processor of the device 110. A wearer can view detailed information and graphical representations of their training zones for an activity on the personal computer 135, mobile device 121, and/or display 130. Alternatively, or in addition, training zone information can be displayed to the user on a digital display 130.

The device 110 can further include a heart rate monitor, and can be configured to store and/or transmit heart rate information over time in correspondence with H⁺ concentration data. Heart rate monitors are known in the art, and a standard form of portable heart rate monitor typically measures electrical signals at the skin, which are a result of electrical activity traveling from the heart. The heart rate may be obtained by measuring a time between signal peaks and then calculating a frequency of the peaks in a given unit of time, for example, beats per minute. The components of a heart rate monitor typically include electrodes, filters and amplifiers to magnify the signal, analog-to-digital converter, microcontroller, clock, voltage regulator, battery, and, generally, a display. The components of a heart rate monitor can be included in a wearable lactate threshold monitor. Heart rate information can be further manipulated in a personal computing device or server. Alternatively, or in addition, the device 110 can include other components to provide additional functionality to the user. For example, a clock, stopwatch, alarm, compass, and/or GPS sensor may be included in a wearable lactate threshold monitor. The device 110 can be configured to store and/or transmit date and time information, heart rate information, and/or location information in correspondence with H+ concentration data.

The device 110 can further include additional imaging-related MR components. For example, a cross-sectional image of an anatomical region located under or within the device may be obtained. Imaging information may be obtained by further processing of the data detected at an RF receive coil (e.g., transmit/receive coil 360 of FIG. 3), using components as previously described (e.g., processor 340, etc. of FIG. 3). Additional cross sectional images, or slices, of sequential regions of interest may also be obtained via the addition of gradient coils. Gradient coils may be loops of wire or thin conductive sheets located on a cylindrical sheet, which can be positioned on the surface of a main static field magnet near to the skin. The coils can be activated by running a current through the wire, which interacts with the magnetic field from the static field magnet. This interaction can cause a distortion of the primary static field, effectively moving it a few millimeters (or centimeters). Manipulation of the voltage within the gradient coils can allow images to be taken of multiple anatomical slices, each separated by millimeters (or centimeters). Imaging related components can be miniaturized to fit within a wearable device. The images may allow visualization of the concentration of hydrogen protons within the tissue under the magnet. As the concentration of hydrogen protons increases (for example, as the wearer passes through and above lactate threshold), the image may allow visualization of this state within the tissue.

The wearer of the device can be a human or an animal, such as a racehorse. The handler of an animal engaged in an athletic activity can provide an improved training plan for the animal by having information regarding the animal's lactate threshold and/or by a real-time indicator of when the animal has reached its lactate threshold.

FIG. 6 depicts a series of athletic training zones 600, which can be used to identify and quantify the degree of physical stress that an athlete may be feeling during a given workout. The response to stress (e.g., physical exertion) can be identified and tracked via one or more modes, including heart rate 610, relative perceived exertion 612 and blood lactate level 630. Relative perceived exertion 612 can be tracked by the user, and technology for monitoring heart rates 610 is known in the art. Training zones 600 can be defined as, for example, a percentage of the athlete's race pace, as indicated in FIG. 6. An athlete's race pace can be defined as the speed or level of physical exertion that the athlete is capable of maintaining for the entirety of an event. Athletic training programs use such information and tracking tools to plan workouts on both a macro and a micro level. A macro level generally refers to weekly and/or monthly cycles, and a micro level generally refers to daily, hourly, and or minutes of work performed.

MR-based wearable lactate threshold monitors can further enable athletic training zone information to be defined based upon lactate levels and lactate threshold, which can provide more precise indications of an athlete's level of physical stress than relative perceived exertion, which is subjectively tracked by the athlete. MR-based wearable devices can provide lactate threshold information, based on measure H⁺ concentration data, in real time and without the inconvenience of blood testing. FIG. 6 illustrates an example of athletic training zone information. As shown in FIG. 6, actual measured values of lactate levels 630 in the blood correspond with different levels of relative perceived exertion 612, as well as heart rate 610. Similar to the use of lactate levels as measured from blood sampling, and the alignment of blood lactate levels with target training zones for an athlete, the plotting of H⁺ concentration data can be used to define training zones 600 and can be further correlated to other indicators, such as heart rate 610 and relative perceived exertion 612.

Typical detailed training plans utilize training zones (e.g., training zones 600), which can be defined based on percentage(s) of the wearer's lactate threshold. Lactate threshold information can thus be used to help the athlete better understand their level(s) of performance and can be used to provide athletes with more customized athletic training zone information. Training zone information 600 can be presented to users on the wearable lactate threshold monitor, for example, on display 130 of device 110 (FIG. 5). Alternatively, training zone information can be presented to the user on remote devices, such as mobile device 121, remote server 125, and/or personal computer 135 (FIG. 5).

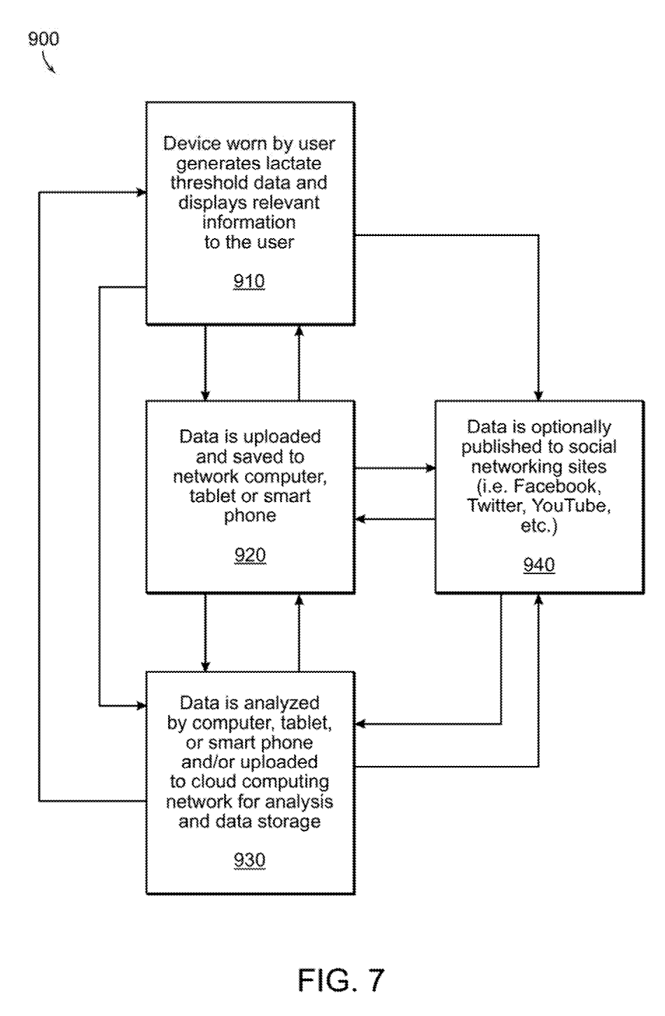

FIG. 7 is a block diagram illustrating a data processing sequence 900 for use with an MR-based wearable lactate threshold monitor. Data, such as H⁺ concentration data, is generated by a wearable lactate threshold monitor and displayed to a user at step 910. Data generated at step 910 can optionally be uploaded and saved to a networked computer, tablet, or smartphone, as shown at step 920. Additional calculations can be performed by the networked computer, tablet, smartphone, or cloud computing network, as shown at step 930. For example, additional calculations can include analysis of H+ concentration data, determination of athletic training zone information and/or generation of customized training plans. Additional data generated during step 930 can be stored on the networked computer, tablet, smartphone, or cloud computing network (step 920) and/or can be returned to the wearable device for display to the user (step 910). Data, including data generated by the device (step 910) and analyzed data generated by networked devices (step 930) can optionally be published to social networking sites, such as Facebook®, Twitter®, YouTube®, and the like (step 940). Further, socially networked data (step 940) can be subsequently transmitted or uploaded to networked devices (step 920) and/or to the device (step 910) for display to the user.

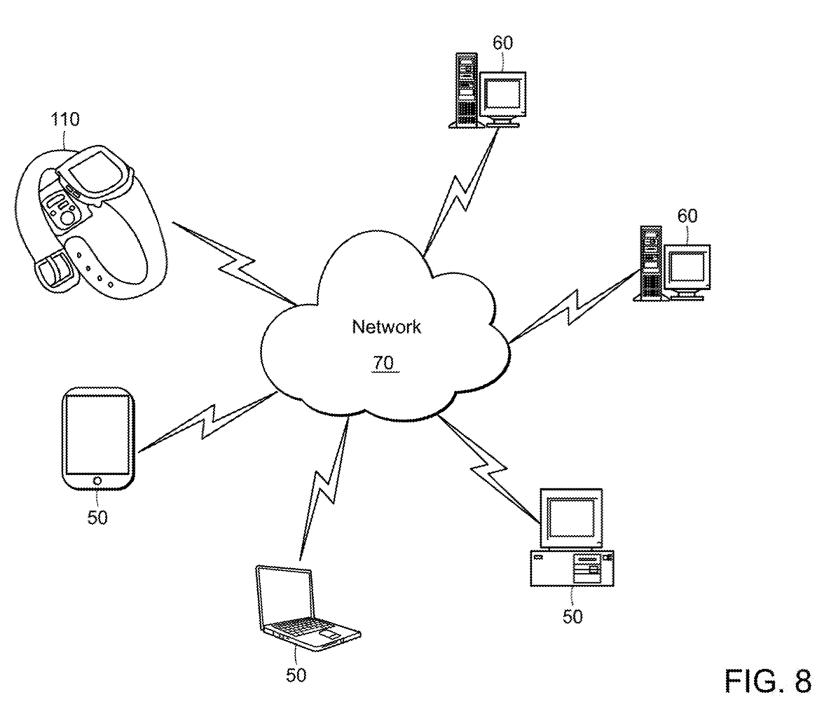

FIG. 8 illustrates a computer network or similar digital processing environment in which embodiments of the present invention may be implemented. Client computer(s)/mobile device(s) 50, wearable device(s) 110, and server computer(s) 60 provide processing, storage, and input/output devices executing application programs and the like. Client computer(s)/devices 50 can also be linked through communications network 70 to other computing devices, including other client devices/processes 50 and server computer(s) 60. Communications network 70 can be part of a remote access network, a global network (e.g., the Internet), a worldwide collection of computers, Local area or Wide area networks, and gateways that currently use respective protocols (TCP/IP, Bluetooth, etc.) to communicate with one another. Other electronic device/computer network architectures are suitable.

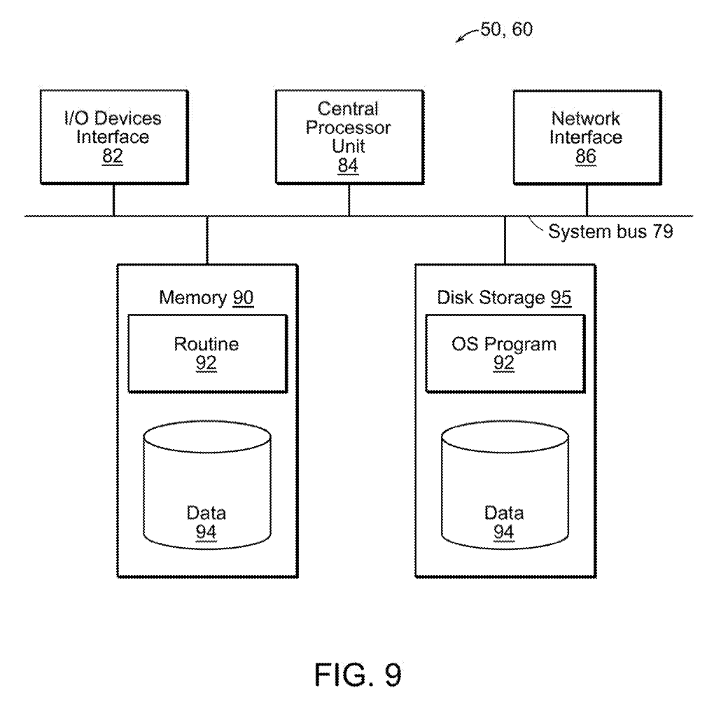

FIG. 9 is a diagram of the internal structure of a computer (e.g., client processor/device 50 or server computers 60) in the computer network of FIG. 8. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, mouse, displays, printers, speakers, etc.) to the computer 50, 60. Network interface 86 allows the computer to connect to various other devices attached to a network (e.g., network 70 of FIG. 2). Memory 90 provides volatile storage for computer software instructions 92 and data 94 used to implement an embodiment of the present invention (e.g., receiving and performing computations relating to the detection of a lactate threshold and calculation of training zones, and further described in the Appendix). Disk storage 95 provides nonvolatile storage for computer software instructions 92 and data 94 used to implement an embodiment of the present invention. Central processor unit 84 is also attached to system bus 79 and provides for the execution of computer instructions.

In one embodiment, the processor routines 92 and data 94 are a computer program product (generally referenced 92), including a non-transitory computer readable medium (e.g., a removable storage medium such as one or more DVD-ROM's, CD-ROM's, diskettes, tapes, etc.) that provides at least a portion of the software instructions for the invention system. Computer program product 92 can be installed by any suitable software installation procedure, as is well known in the art. In another embodiment, at least a portion of the software instructions may also be downloaded over a cable, communication and/or wireless connection. In other embodiments, the invention programs are a computer program propagated signal product 107 embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present invention routines/program 92.

In alternative embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, other mediums and the like.

Alternative embodiments can include or employ clusters of computers, parallel processors, or other forms of parallel processing, effectively leading to improved performance, for example, of generating a computational model.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A wearable device for monitoring a lactate threshold, the device comprising:
at least one magnet configured to provide a static magnetic field to an anatomical region;
a power supply; and
a radio frequency (RF) module connected to the power supply for power, the RF module configured to provide pulsating RF signals across the static magnetic field and emit energy into the anatomical region over a period of time, and further configured to receive response signals from the anatomical region in response to the energy emitted into the anatomical region over the period of time, the response signals enabling detection of a change in H⁺ concentration in the anatomical region, the change enabling detection of the lactate threshold.

2. The wearable device of claim 1, wherein the period of time is at least one of the following: between thirty seconds and one hour, or between an hour and twenty-four hours.

3. The wearable device of claim 1, wherein the magnet is an open magnet.

4. The wearable device of claim 1, wherein the magnet is a closed magnet.

5. The wearable device of claim 1, further comprising a processing component coupled to the RF module and configured to calculate a moving average of H⁺ concentration within the anatomical region.

6. The wearable device of claim 1, further comprising a processing component coupled to the RF module and configured to adjust the energy emitted into the anatomical region based on a detected response signal.

7. The wearable device of claim 1, wherein the RF module includes an RF signal generator, RF transmit coil, and RF receive coil, the wearable device further comprising a housing containing the at least one magnet, RF signal generator, RF transmit coil, RF receive coil, and power supply.

8. The wearable device of claim 1, further comprising a Faraday cage encapsulating the magnet and the RF module.

9. The wearable device of claim 1, wherein the magnet has a magnetic flux density of 0.1 T to 2.0 T.

10. The wearable device of claim 1, wherein the magnet has a magnetic flux density of 0.25 T to 0.5 T.

11. The wearable device of claim 1 wherein the device is in the form of one of the following wearable devices: a wristwatch, anklet, or armband.

12. The wearable device of claim 1 wherein the power supply is a rechargeable battery.

13. The wearable device of claim 1, further comprising a wireless transmission component communicatively coupled to the RF module and configured to transmit H⁺ concentration data to a server via a network.

14. The wearable device of claim 1, further comprising:
a processor coupled to the RF module and configured to generate a representation of a change in H+ concentration; and
a display configured to display a detected change in H+ concentration.

15. The wearable device of claim 14, wherein the display is further configured to display athletic training zone information transmitted to the wearable device from a server.

16. The wearable device of claim 1, further comprising a data storage device configured to store H⁺ concentration data and further comprising a data transmitter module configured to connect with a personal computer to download the stored H⁺ concentration data.

17. The wearable device of claim 1, wherein the device further comprises a feature of at least one of the following devices: a heart rate monitor, pedometer, altimeter, thermometer or clock.

18. A method of determining a lactate threshold, comprising:
applying a magnetic field to an anatomical region, the magnetic field provided by at least one magnet within a wearable device;
emitting pulsating RF signals into the anatomical region over a period of time, the RF signals provided by an RF module;
receiving response signals from the anatomical region by the RF module;
measuring concentrations of H⁺ within the anatomical region over the period of time based upon the response signals; and
determining a lactate threshold based upon a detected change in the concentration of H⁺ within the anatomical region.

19. The method of claim 18, wherein the period of time is at least one of the following: between thirty seconds and one hour, or between an hour and twenty-four hours.

20. The method of claim 18, further comprising operating the RF module within a housing.

21. The method of claim 20, further comprising maintaining a position of the housing within proximity of a limb.

22. The method of claim 18, further comprising transmitting measurements of the concentration of H⁺ within the anatomical region to a server via a network.

23. The method of claim 18, further comprising displaying a detected change in the concentration of H⁺.

24. A system comprising a server in a network, the server having a software routine operable thereon, the software routine, when executed, causes the server to perform at least the following:
H⁺ concentration data from the wearable device of claim 1;
calculate athletic training zones based on percentages of the H⁺ concentration data; and
transmit athletic training zone information to the wearable device or a different device.

25. The system of claim 24, wherein the software routine is further configured to cause the server to transmit the athletic training zone information to a personal computer, tablet, smartphone, or personal digital assistant.
